(19) United States
(12) Patent Application Publication
(10) Pub. No.: US 2012/0028906 A1
(43) Pub. Date: Feb. 2, 2012

(54) METHOD FOR AFFECTING THE TIMING OF CERVICAL RIPENING AND FOR PARTURITION

(75) Inventor: Steven M. Yellon, Redlands, CA (US)

(73) Assignee: LOMA LINDA UNIVERSITY, Loma Linda, CA (US)

(21) Appl. No.: 13/146,192

(22) PCT Filed: Mar. 10, 2010
(86) PCT No.: PCT/US10/26875

§ 371 Date: Jul. 25, 2011

Related U.S. Application Data
(60) Provisional application No. 61/160,138, filed on Mar. 13, 2009.

(51) Int. Cl. A61K 38/08; A61K 31/4025; A61K 38/17; A61P 15/04; A61K 31/167; A61K 31/439; A61K 31/13; A61K 31/465; A61K 31/165

(52) U.S. Cl. 514/18.1; 514/343; 514/422; 514/17.7; 514/627; 514/626; 514/304; 514/661; 514/292

(57) ABSTRACT

A method for affecting the timing of cervical ripening and the resultant onset of parturition in a pregnant female mammal, the method comprises administering to a pregnant female one or more than one dose of an agent that chemically mimics innervation of the cervix by the vagus nerve, thereby advancing the onset of parturition, or administering to a pregnant female one or more than one dose of an agent that chemically blocks innervation of the cervix by the vagus nerve, thereby delaying the onset of parturition.

### CROSS-REFERENCE TO RELATED APPLICATION

This application claims the benefit of U.S. Provisional Patent Application No. 61/160,138, entitled “Method for Affecting the Timing of Cervical Ripening and for Parturition,” filed Mar. 13, 2009, the contents of which are incorporated by reference herein in its entirety.

### STATEMENT REGARDING FEDERALLY SPONSORED RESEARCH OR DEVELOPMENT

This invention was made with United States Government support under National Institutes of Health Grant No. R01-HD054931. The United States Government has certain rights in this invention.

### BACKGROUND

Normal parturition in a pregnant female mammal requires complete ripening and dilation of the cervix. Failure of complete ripening and dilation of the cervix to occur contributes to fetal distress during the birth process. Additionally, failure of complete ripening and dilation of the cervix to occur contributes, in part, to increasing rates of Cesarean section deliveries to circumvent these failures, in both the United States and in other countries. By contrast, premature ripening and dilation of the cervix leads to preterm labor, which is the leading obstetrical and pediatric problem in the United States, and which accounts for 70% of all neonatal mortality before 37 weeks of gestation.

Therefore, there is a need for a method for affecting the timing of cervical ripening and for parturition.

### SUMMARY

According to one embodiment of the present invention, there is provided a method for affecting the timing of cervical ripening, thereby advancing the onset of parturition in a pregnant female mammal. In one embodiment, the method comprises a) determining the degree of cervical ripening in the pregnant female mammal, where the degree determined is a first level of cervical ripening; b) administering to the pregnant female mammal one or more than one dose of an agent that chemically mimics innervation of the cervix by the vagus nerve, or administering to the pregnant female mammal one or more than one dose of a composition comprising one or more than one agent that chemically mimics innervation of the cervix by the vagus nerve; and c) determining the degree of cervical ripening in the pregnant female mammal, where the degree determined is a second level of cervical ripening; and where the first level of cervical ripening is less than the second level of cervical ripening, thereby advancing the onset of parturition in a pregnant female mammal.

In one embodiment, the pregnant female mammal is a human. In another embodiment, the agent is a cholinergic agonist. In a preferred embodiment, the cholinergic agonist is a non-selective muscarinic receptor agonist. In a particularly preferred embodiment, the non-selective muscarinic receptor agonist is selected from the group consisting of bromocytisine, nicotine and oxotremorine (1-(4-pyrrolidin-1-ylbut-2-yn-1-yl) pyrrolidin-2-one). In one embodiment, the agent is a sensory agonist. In a preferred embodiment, the sensory agonist is selected from the group consisting of substance P (SP) and calcitonin gene-related peptide (CGRP). In one embodiment, the composition comprises an agent that is a cholinergic agonist, and further comprises an agent that is a sensory agonist.

According to one embodiment of the present invention, there is provided a method for affecting the timing of cervical ripening, thereby delaying the onset of parturition in a pregnant female mammal. The method comprises a) determining the degree of cervical ripening in the pregnant female mammal, where the degree determined is a first level of cervical ripening; b) administering to the pregnant female mammal one or more than one dose of an agent that chemically blocks innervation of the cervix by the vagus nerve, or administering to the pregnant female mammal one or more than one dose of a composition comprising one or more than one agent that chemically blocks innervation of the cervix by the vagus nerve; and c) determining the degree of cervical ripening in the pregnant female mammal, where the degree determined is a second level of cervical ripening; and where the first level of cervical ripening is more than the second level of cervical ripening, thereby delaying the onset of parturition in a pregnant female mammal.

In one embodiment, the pregnant female mammal is a human. In another embodiment, the agent is a cholinergic antagonist. In a preferred embodiment, the cholinergic antagonist is selected from the group consisting of atropine and mecamylamine. In another embodiment, the agent is a sensory antagonist. In a preferred embodiment, the sensory antagonist is selected from the group consisting of capsaicin, lidocaine and a selective NK1 receptor antagonist (such as, for example, L-703606, available from Sigma-Aldrich Co., St. Louis, Mo. US). In one embodiment, the composition comprises an agent that is a cholinergic antagonist, and further comprises an agent that is a sensory antagonist.

According to one embodiment of the present invention, the dose of the agent administered is between about 0.1 and 10,000 micrograms per kilogram of body weight or environmental medium. In another embodiment, the dose of the agent administered is between about 1 and 1,000 micrograms per kilogram of body weight or environmental medium. In another embodiment, the dose of the agent administered, whether administered alone or in a composition, is between about 10 and 1,000 micrograms per kilogram of body weight or environmental medium. In another embodiment, the dose is preferably administered in a final volume of between about 0.1 and 10 ml. In another embodiment, the dose is administered in a final volume of between about 0.01 and 1 ml.

According to one embodiment of the present invention, there is provided a method for affecting the timing of cervical ripening, thereby advancing the onset of parturition in a pregnant female mammal, where the method comprises administering to the pregnant female mammal one or more than one dose of an agent that chemically mimics innervation of the cervix by the vagus nerve, or administering to the pregnant female mammal one or more than one dose of a composition comprising one or more than one agent that chemically mimics innervation of the cervix by the vagus nerve.

According to one embodiment of the present invention, there is provided a method for affecting the timing of cervical ripening, thereby delaying the onset of parturition in a pregnant female mammal, where the method comprises administering to the pregnant female mammal one or more than one dose of an agent that chemically blocks innervation of the cervix by the vagus nerve, or administering to the pregnant female mammal one or more than one dose of a composition comprising one or more than one agent that chemically blocks innervation of the cervix by the vagus nerve.

### DESCRIPTION

According to one embodiment of the present invention, there is provided a method for affecting the timing of cervical ripening and the resultant onset of parturition in a pregnant female mammal. In one embodiment, the method comprises administering to a pregnant female one or more than one dose of an agent that chemically mimics innervation of the cervix by the vagus nerve, thereby advancing the onset of parturition. In one embodiment, the method comprises administering to the pregnant female one or more than one dose of a composition comprising one or more than one agent that chemically mimics innervation of the cervix by the vagus nerve, thereby advancing the onset of parturition. In another embodiment, the method comprises administering to a pregnant female one or more than one dose of an agent that chemically blocks innervation of the cervix by the vagus nerve, thereby delaying the onset of parturition. In one embodiment, the method comprises administering to the pregnant female one or more than one dose of a composition comprising one or more than one agent that chemically blocks innervation of the cervix by the vagus nerve, thereby delaying the onset of parturition. In a preferred embodiment, the pregnant female is a human pregnant female. The vagus nerve is an extra-spinal cord sensory afferent projection to the cervix comprising both cholinergic and sensory innervation.

As used herein, except where the context requires otherwise, the term “comprise” and variations of the term, such as “comprising,” “comprises” and “comprised” are not intended to exclude other additives, components, integers or steps.

As used in this disclosure, the level of cervical ripening is determined according to standard techniques as will be understood by those with skill in the art with reference to this disclosure, such as for example, by cervico-vaginal fluid analyses, ultrasound, and pelvic examinations.

As used in this disclosure, the term “administering” includes any suitable route of administration, as will be appreciated by one of ordinary skill in the art with reference to this disclosure, including direct injection, inhalation, intraperitoneal injection, intravenous injection, parenteral, topical application on a mucous membrane, or application to or dispersion within an environmental medium, and a combination of the preceding, or by any other suitable method. In a preferred embodiment, administering comprises using an intravaginal route, such as for example intravaginal application of a gel to provide either an acute dose of the agent or timed release dose. Intravaginal application can be performed using a cervical ring or a diaphragm, or other suitable device, as will be understood by those with skill in the art with reference to this disclosure.

Remodeling of the cervix is associated with breakdown of collagen, inflammation, and increased innervation in mammals, including both rodents and humans. In one embodiment, the present invention is the discovery that the cervix is innervated by the vagus nerve (in addition to innervation by other nerves that were previously known to innervate the cervix). Further, innervation by the vagus nerve affects both the level of cervical ripening and the timing of parturition. These effects were demonstrated as follows.

On day 15 post-breeding, pregnant Long Evans rats had a subdiaphragm segment of the vagus nerve transected (VnX, n=5) according to IACUC (Institutional Animal Care and Use Committee) approved procedures. Groups of pregnant controls were either sham-operated (Sham, n=4) or sham-operated nonpregnant (NP, n=3). The pregnant rats were killed prepartum (day 21 post-breeding) and postpartum (day of birth). The cervices were excised, fixed in 4% paraformaldehyde, and processed to stain collagen with picrosirius red or by immunohistochemistry to identify macrophages or nerve fibers.

The results of the research were that the VnX had delayed parturition, giving birth from the evening of day 22 to the morning of day 24. By contrast, the Sham control rats gave birth on the morning of day 22. Further, the cervices of the VnX rats showed reduced cell nuclei density, as well as collagen content and structure compared to the cervices of both the Sham group before or after birth, and compared to the cervices of the NP controls, however, there were increased numbers of macrophages in the area with nerve fibers in both VnX and Sham rats, as compared to NP rats.

Therefore, as can be appreciated from these data, the vagus nerve regulates the level of cervical ripening thereby affecting timing of parturition Immigration of macrophages and hypertrophy of innervation coincided with remodeling of the cervices of both normal and delayed birth animals as found in PGF2a receptor knock out mice. These results demonstrate that innervation by the vagus nerve promotes cervical ripening leading to parturition, while the lack of innervation by the vagus nerve delays cervical ripening leading to delayed parturition.

According to one embodiment of the present invention, there is provided a method for affecting the timing of cervical ripening, thereby advancing the onset of parturition in a pregnant female mammal. In one embodiment, the pregnant female mammal is a human. In one embodiment, the method comprises administering to the pregnant female one or more than one dose of an agent that chemically mimics innervation of the cervix by the vagus nerve. In one embodiment, the agent is a cholinergic agonist. In another embodiment, the agent is a sensory agonist. In another embodiment, the method comprises administering to the pregnant female one or more than one dose of a composition comprising one or more than one agent that chemically mimics innervation of the cervix by the vagus nerve. In one embodiment, one of the one or more than one agent in the composition is a cholinergic agonist. In another embodiment, one of the one or more than one agent in the composition is a sensory agonist. In another embodiment, one of the one or more than one agent in the composition is a cholinergic agonist, and one of the one or more than one agent in the composition is a sensory agonist. In one embodiment, the cholinergic agonist is a non-selective muscarinic receptor agonist selected from the group consisting of bromocytisine, nicotine and oxotremorine (1-(4-pyrrolidin-1-ylbut-2-yn-1-yl)pyrrolidin-2-one). In another embodiment, the sensory agonist is selected from the group consisting of substance P (SP) and calcitonin gene-related peptide (CGRP).

In one embodiment, the present invention is a method for affecting the timing of cervical ripening, thereby advancing the onset of parturition in a pregnant female mammal. The method comprises a) determining the degree of cervical ripening in the pregnant female mammal, where the degree determined is a first level of cervical ripening; b) administering to the pregnant female mammal one or more than one dose of an agent that chemically mimics innervation of the cervix by the vagus nerve, or administering to the pregnant female mammal one or more than one dose of a composition comprising one or more than one agent that chemically mimics innervation of the cervix by the vagus nerve; and c) determining the degree of cervical ripening in the pregnant female mammal, where the degree determined is a second level of cervical ripening; and where the first level of cervical ripening is less than the second level of cervical ripening, thereby advancing the onset of parturition in a pregnant female mammal.

According to one embodiment of the present invention, there is provided a method for affecting the timing of cervical ripening, thereby delaying the onset of parturition in a pregnant female mammal. In one embodiment, the pregnant female mammal is a human. In one embodiment, the method comprises administering to a pregnant female one or more than one dose of an agent that chemically blocks innervation of the cervix by the vagus nerve. In another embodiment, the method comprises administering to the pregnant female one or more than one dose of a composition that comprises an agent that chemically blocks innervation of the cervix by the vagus nerve. In one embodiment, the agent is a cholinergic antagonist. In another embodiment, the agent is a sensory antagonist. In another embodiment, the method comprises administering to the pregnant female one or more than one dose of a composition comprising one or more than one agent that chemically blocks innervation of the cervix by the vagus nerve. In one embodiment, one of the one or more than one agent in the composition is a cholinergic antagonist. In another embodiment, one of the one or more than one agent in the composition is a sensory antagonist. In another embodiment, the composition comprises both a cholinergic antagonist and a sensory antagonist. In one embodiment, the cholinergic antagonist is selected from the group consisting of atropine and mecamylamine In one embodiment, the sensory antagonist is selected from the group consisting of capsaicin, lidocaine and a selective NK1 receptor antagonist (such as, for example, L-703606, available from Sigma-Aldrich Co., St. Louis, Mo. US)

In another embodiment, the present invention is a method for affecting the timing of cervical ripening, thereby delaying the onset of parturition in a pregnant female mammal. The method comprises a) determining the degree of cervical ripening in the pregnant female mammal, where the degree determined is a first level of cervical ripening; b) administering to the pregnant female mammal one or more than one dose of an agent that chemically blocks innervation of the cervix by the vagus nerve, or administering to the pregnant female mammal one or more than one dose of a composition comprising one or more than one agent that chemically blocks innervation of the cervix by the vagus nerve; and c) determining the degree of cervical ripening in the pregnant female mammal, where the degree determined is a second level of cervical ripening; and where the first level of cervical ripening is more than the second level of cervical ripening, thereby delaying the onset of parturition in a pregnant female mammal.

In one embodiment, the dose of the agent administered, whether administered alone or in a composition, is between about 0.1 and 10,000 micrograms per kilogram of body weight or environmental medium. In another embodiment, the dose of the agent administered, whether administered alone or in a composition, is between about 1 and 1,000 micrograms per kilogram of body weight or environmental medium. In another embodiment, the dose of the agent administered, whether administered alone or in a composition, is between about 10 and 1,000 micrograms per kilogram of body weight or environmental medium. For intravenous injection and intraperitoneal injection, the dose is preferably administered in a final volume of between about 0.1 and 10 ml. For inhalation the dose is preferably administered in a final volume of between about 0.01 and 1 ml. As will be appreciated by one of ordinary skill in the art with reference to this disclosure, the dose can be repeated at one or more than one of times as needed using the same parameters to effect the purposes disclosed in this disclosure.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference in their entirety.

1. A method for affecting the timing of cervical ripening, thereby advancing the onset of parturition in a pregnant female mammal, the method comprising:
a) determining the degree of cervical ripening in the pregnant female mammal, where the degree determined is a first level of cervical ripening;
b) administering to the pregnant female mammal one or more than one dose of an agent that chemically mimics innervation of the cervix by the vagus nerve, or administering to the pregnant female mammal one or more than one dose of a composition comprising one or more than one agent that chemically mimics innervation of the cervix by the vagus nerve; and
c) determining the degree of cervical ripening in the pregnant female mammal, where the degree determined is a second level of cervical ripening; and
where the first level of cervical ripening is less than the second level of cervical ripening, thereby advancing the onset of parturition in a pregnant female mammal.

2. The method of claim 1, where the pregnant female mammal is a human.

3. The method of claim 1, where the agent is a cholinergic agonist.

4. The method of claim 3, where the cholinergic agonist is a non-selective muscarinic receptor agonist.

5. The method of claim 4, where the non-selective muscarinic receptor agonist is selected from the group consisting of bromocytisine, nicotine and oxotremorine (1-(4-pyrrolidin-1-ylbut-2-yn-1-yl)pyrrolidin-2-one).

6. The method of claim 1, where the agent is a sensory agonist.

7. The method of claim 6, where the sensory agonist is substance P (SP) and calcitonin gene-related peptide (CGRP).

8. The method of claim 1, where the composition comprises an agent that is a cholinergic agonist, and further comprises an agent that is a sensory agonist.

9. A method for affecting the timing of cervical ripening, thereby delaying the onset of parturition in a pregnant female mammal, the method comprising:
a) determining the degree of cervical ripening in the pregnant female mammal, where the degree determined is a first level of cervical ripening;
b) administering to the pregnant female mammal one or more than one dose of an agent that chemically blocks innervation of the cervix by the vagus nerve, or administering to the pregnant female mammal one or more than one dose of a composition comprising one or more than one agent that chemically blocks innervation of the cervix by the vagus nerve; and
c) determining the degree of cervical ripening in the pregnant female mammal, where the degree determined is a second level of cervical ripening; and
where the first level of cervical ripening is more than the second level of cervical ripening, thereby delaying the onset of parturition in a pregnant female mammal.

10. The method of claim 9, where the pregnant female mammal is a human.

11. The method of claim 9, where the agent is a cholinergic antagonist.

12. The method of claim 11, where the cholinergic antagonist is selected from the group consisting of atropine and mecamylamine

13. The method of claim 9, where the agent is a sensory antagonist.

14. The method of claim 13, where the sensory antagonist is selected from the group consisting of capsaicin, lidocaine and a selective NK1 receptor antagonist.

15. The method of claim 9, where the composition comprises an agent that is a cholinergic antagonist, and further comprises an agent that is a sensory antagonist.

16. The method of claim 1, where the dose of the agent administered is between about 0.1 and 10,000 micrograms per kilogram of body weight or environmental medium.

17. The method of claim 1, where the dose of the agent administered is between about 1 and 1,000 micrograms per kilogram of body weight or environmental medium.

18. The method of claim 1, where the dose of the agent administered, whether administered alone or in a composition, is between about 10 and 1,000 micrograms per kilogram of body weight or environmental medium.

19. The method of claim 1, where the dose is preferably administered in a final volume of between about 0.1 and 10 ml.

20. The method of claim 1, where the dose is administered in a final volume of between about 0.01 and 1 ml.

21. A method for affecting the timing of cervical ripening, thereby advancing the onset of parturition in a pregnant female mammal, where the method comprises administering to the pregnant female mammal one or more than one dose of an agent that chemically mimics innervation of the cervix by the vagus nerve, or administering to the pregnant female mammal one or more than one dose of a composition comprising one or more than one agent that chemically mimics innervation of the cervix by the vagus nerve.

22. A method for affecting the timing of cervical ripening, thereby delaying the onset of parturition in a pregnant female mammal, where the method comprises administering to the pregnant female mammal one or more than one dose of an agent that chemically blocks innervation of the cervix by the vagus nerve, or administering to the pregnant female mammal one or more than one dose of a composition comprising one or more than one agent that chemically blocks innervation of the cervix by the vagus nerve.

23. The method of claim 9, where the dose of the agent administered is between about 0.1 and 10,000 micrograms per kilogram of body weight or environmental medium.

24. The method of claim 9, where the dose of the agent administered is between about 1 and 1,000 micrograms per kilogram of body weight or environmental medium.

25. The method of claim 9, where the dose of the agent administered, whether administered alone or in a composition, is between about 10 and 1,000 micrograms per kilogram of body weight or environmental medium.

26. The method of claim 9, where the dose is preferably administered in a final volume of between about 0.1 and 10 ml.

27. The method of claim 9, where the dose is administered in a final volume of between about 0.01 and 1 ml.
